(19) United States
(12) Patent Application Publication
(10) Pub. No.: US 2006/0029678 A1
(43) Pub. Date: Feb. 9, 2006

(54) Process for the production of implants

(76) Inventor: Romano Deghenghi, St. Cergue (CH)

Correspondence Address: WINSTON & STRAWN LLP, 1700 K STREET, N.W., WASHINGTON, DC, 20006, US

(21) Appl. No.: 11/215,031

(22) Filed: Aug. 29, 2005

Related U.S. Application Data
(63) Continuation of application No. PCT/GB04/00816, filed on Mar. 1, 2004.

(30) Foreign Application Priority Data
Mar. 1, 2003 (GB) GB 0304726.3

(51) Int. Cl. A61K 9/14; A61K 38/17

(52) U.S. Cl. 424/489; 514/2

(57) ABSTRACT

There is provided a process for the preparation of an implantable or injectable pharmaceutical composition suitable for the extended release of an active ingredient, such as a peptide or a peptide analogue, to a patient following administration, by a process that includes: (a) wet granulation of a mixture of active ingredient and PLGA; (b) drying the granules so formed; (c) grinding the dried granules; and (d) extruding the ground product of step (c).

### CROSS REFERENCE TO RELATED APPLICATIONS

This application is a continuation of International Application PCT/GB2004/000816 filed Mar. 1, 2004, the entire content of which is expressly incorporated herein by reference thereto.

### BACKGROUND

This invention relates to a novel process for the production of pharmaceutical compositions suitable for use as, or in the manufacture of, inter alia subcutaneous implants.

It is often desirable to release pharmaceutically-active compounds, in particular bioactive peptides and peptide analogues, over an extended period of time.

Compositions that provide for a modified release of pharmaceutically-active compounds are well known in the art. Indeed, over the last thirty or so years, modified release dosage forms have increasingly become a preferred method of delivering certain drugs to patients.

In the case of peptides, extended release drug delivery systems based on microparticles (such as microspheres, microcapsules and microgranules), and implant forms for intramuscular or subcutaneous injection have been well documented.

Sustained release implant systems in the form of peptide/peptide analogues dispersed in a cylinder of bioerodible polymer are known to give rise to an extended release of such active materials. In particular, implants in which active ingredient is dispersed in a polymer matrix comprising lactic acid and/or gycolic acid units have been described in inter alia U.S. Pat. No. 5,366,734. However, the maximum time observed for the release of active ingredient from implants prepared in accordance with the procedures described in U.S. Pat. No. 5,366,734 is in the order of three months.

U.S. Pat. No. 5,456,917 discloses a process for forming an implantable bioerodible composition for the sustained release of a medicament comprising the steps of grinding a copolymer of lactic acid and glycolic acid (PLGA), selecting particles of a pre-determined size, dissolving the medicament in a solvent in which the PLGA is not soluble, adding the PLGA particles to that solution, applying and releasing a vacuum to load the pores of the PLGA particles with the solution, isolating the loaded polymer particles by filtration or decanting, and then freeze-drying or vacuum-drying to remove the residual solvent from within the pores. The blended mixture is then placed in an extrusion device to form implants.

UK patent application 2,234,169 discloses a method for preparing a sustained release pharmaceutical peptide composition comprising a PLGA copolymer and a salt of a natural or synthetic water insoluble peptide.

International patent application WO 97/41836 discloses implantable pharmaceutical compositions for the sustained release of an active agent over a period of more than one month, comprising a homopolymer of lactic acid in association with an insoluble salt of a protein, polypeptide or hormone.

International patent application WO 98/09613 describes a process for the preparation of subcutaneous implants comprising bioactive peptides and PLGA, which process comprises grinding a PLGA copolymer, wetting the copolymer with an aqueous slurry of a peptide, mixing this copolymer and slurry so as to obtain a homogeneous mixture, drying the mixture and then extruding in order to obtain implants for subcutaneous use.

Finally, international patent application WO 00/33809 discloses a process for obtaining extended release implants comprising peptide particles distributed heterogeneously throughout a PLGA matrix. This process involves the essential steps of homogeneously mixing peptide in the form of particles having a diameter of between 1 and 60 μm when dry with PLGA, wet granulation of the resultant mixture, drying of the granulate to a residual liquid content of between 0.5 to 2.0% by weight, and then extruding the dried granulate to produce implants.

However, there remains a general need for pharmaceutical implants that are capable of maintaining an extended release of drugs, such as peptides and peptide analogues, over a period of, for example, three to six months (or in some cases even longer), and improved processes for making the same.

Nowhere in WO 00/33809, or any of the other prior art documents mentioned hereinbefore, is a process described for the manufacture of subcutaneous implants, which process comprises the essential step of grinding pre-formed granules comprising a mixture of active ingredient and PLGA prior to the extrusion of the ground material into a form that can be cut into cylindrical implants.

### SUMMARY OF THE INVENTION

According to the invention, there is provided a process for the preparation of an implantable or injectable pharmaceutical composition suitable for the extended release of an active ingredient, such as a peptide or a peptide analogue, to a patient following administration, which process comprises: (a) wet granulation of a mixture of an active ingredient and PLGA; (b) drying the granules so formed; (c) grinding the dried granules; and (d) extruding the ground product of step (c), which process is hereinafter referred to as “the process of the invention”.

### DETAILED DESCRIPTION OF THE PREFERRED EMBODIMENTS

The term “peptide analogue” will be understood by those skilled in the art to include any structurally-similar compound that has the same or similar biological function or activity to a biologically-active peptide.

The extruded product of the process of the invention may, if necessary, be cut into appropriate lengths so as to form implants for intramuscular or, preferably, subcutaneous administration. Appropriate lengths of implants suitable for such administration are in the range of about 10 to about 60 mm, for example, 12 to 55 mm, such as 15 to 50 mm, preferably 18 to 45 mm and, more preferably, 20 to 40 mm. Depending on the diameter of the implants, and the active ingredient, that is/are employed, preferred implant lengths are in the range 24 to 38 mm, such as about 26.6 mm, about 33 mm, about 35 mm or about 37 mm.

The extruded product of the process of the invention may also be sterilized using standard techniques and equipment. Sterilization may be conducted prior to, or after, packaging of the extruded product and/or implants obtained therefrom. Suitable packaging materials include aluminum pouches. Implants prepared by way of the process of the invention may also be presented in the needle of a syringe, from which it is intended to deliver the implant subcutaneously to a patient. Suitable syringes that may be so employed include those described in European patent No. EP 749,336 or U.S. Pat. No. 5,810,769. Syringes and implants (both of which may be pre-sterilized) may be brought into association with each other using known techniques, packaged in standard packaging materials (such as aluminum pouches) and, thereafter, and if appropriate, sterilized using standard techniques/equipment.

The process of the invention may be used to provide compositions which are substantially cylindrical in shape and are of a diameter of between about 1 and about 3 mm, such as between 1.2 and 2.5 mm, preferably 1.4 to 2.2 mm, and more preferably 1.5 to 2 mm (such as about 1.6 mm, about 1.8 mm or about 2 mm). In this respect, in the case of implants, depending upon the active ingredient that is employed and the dose that it is intended to administer, preferred implant dimensions may include diameters of about 1.6 mm and lengths of either about 33 mm or about 35 mm, diameters of about 1.8 mm and lengths of about 37 mm, and diameters of about 2.0 mm and lengths of about 26.6 mm. It will be appreciated that all of the above-mentioned preferred dimensions are approximate, and that implants with lengths/diameters which vary from those numbers specified above by ±20%, such as ±10%, e.g.,±5% are intended to be encompassed by the use of the term “about”. In any event, suitable implant dimensions, which will depend upon inter alia the raw materials that are employed and the dose of active material that it is intended to administer, may be determined routinely by the skilled person.

Suitable PLGA copolymers for use in the process of the invention may have a molar ratio of lactic acid to glycolic acid monomers in the region of 40:60 to 95:5, preferably 60:40 to 90:10, and more preferably 70:30 to 80:20, such as about 75:25. Suitable molecular weights (e.g., number average, z-average or weight average molecular weights, as determined by, for example, ultracentrifugation, light scattering, intrinsic viscosity measurements or, preferably, gel permeation chromatography) are in the range of about 50,000 to about 150,000, preferably 75,000 to 150,000.

It is preferred that PLGA in the form of particles with sizes in the range of about 30 μm to about 200 μm, such as about 40 μm to about 175 μm, and particularly in the range of about 50 μm to about 150 μm, are employed in the wet granulation step of the process of the invention. In this respect, PLGA may be pre-prepared for wet granulation by way of one or more grinding steps. Again, it will be appreciated that all of the above-mentioned preferred particle sizes are approximate and that sizes which vary from those specified above by ±20%, such as +10%, e.g., ±5% are intended to be encompassed by the use of the term “about”. The grinding steps may be performed under cryogenic conditions (such as between 10 and 15° C., e.g., about 12° C.) using standard grinding apparatus, for example as described hereinafter. Appropriate lubricating agents, such as ethanol, may be employed in such polymer grinding. Appropriate fractions of pre-ground polymer may be collected using standard techniques, such a sieving, for example as described hereinafter. PLGA may also be dried prior to wet granulation.

Active ingredients that may be employed in the process of the invention include GNR-H (LHRH), growth hormone releasing hormone, growth hormone releasing peptides, angiotensin, bombesin, bradykinin, cholecystokinin, enkephalin, neurokinin or tachykinin, or agonists or antagonists of the receptors of any of these. Active ingredients that may be employed also include renin inhibitors, protease inhibitors, metallopeptidase inhibitors, enkephalinase inhibitors or atrial or brain natriuretic factor degrading enzyme inhibitors. More specific active ingredients that may be employed include buserelin, deslorelin, histrelin, avorelin, tryptorelin, goserelin, leuprorelin, cetrorelix, teverelix, ramorelix, antide, nictide, azaline B, azaline C, ganirelix or hexarelin. More preferred active ingredients include hexarelin, avorelin, tryptorelin, goserelin and, particularly, leuprorelin and pharmaceutically acceptable salts thereof.

Suitable pharmaceutically-acceptable salts of leuprorelin include pamoate salts, gluconate salts, lactate salts and, preferably, acetate salts.

When the active ingredient that is employed is leuprorelin, and in particular leuprorelin acetate, the weight ratio of leuprorelin (calculated as the weight of the free base, excluding any weight resulting from the presence of a counter ion) to polymer for use in the wet granulation step of the process of the invention is in the region of about 1:10 to about 1:2 w/w, preferably 1:5 to about 1:2.5, such as about 1:4 to about 1:2.75 and particularly about 1:3.5 to about 1:2.85, such as about 1:3 w/w (i.e., 25% leuprorelin w/w). Again, it will be appreciated that all of the above-mentioned preferred weight ratios are approximate and that ratios which vary from those specified above by ±20%, such as ±10%, e.g.,±5% are intended to be encompassed by the use of the term “about”. In any event, suitable weight ratios of raw materials will depend upon inter alia the dose of active ingredient that it is intended to deliver to the patient, as well as the dimensions of the implants that it is intended to produce. However, these may be determined routinely by the skilled person.

Active ingredients may be pre-treated, for example by way of a grinding, or densification, step, prior to the wet granulation step. This may take place in an appropriate apparatus, for example in a ball mill and/or other standard pulverization equipment, for example as described hereinafter.

In any event, polymer and active ingredient are preferably dry mixed (i.e., in the substantial absence of liquid solvents) prior to wet granulation under standard conditions, and in standard mixing equipment, for example as described hereinafter. By “substantial absence” of liquid solvents, we include that no more than 2% (w/w) of liquid solvent (organic or aqueous) is present in any dry mixing step that may be performed prior to wet granulation. Dry mixing is preferably carried out in a ball mill and/or a standard mixer, such as a Turbula mixer or the like, for example as described hereinafter.

Wet granulation may take place under standard conditions and using standard equipment, well known to those skilled in the art, using a suitable liquid, such as ethanol or, preferably, water. When water is employed, it is added to a volume of between 20% and 25%, for example 22% or thereabouts of the total weight of the active/polymer mixture. For example, if 100 g of mixture is employed, 22 mL of water is added prior to granulation. Water may be added prior to granulation in portions to ensure homogeneous mixing with the active/polymer mixture. Standard mixing equipment may be employed to ensure homogeneous mixing, for example as described hereinafter. Wet granulation may thereafter be performed using standard granulation equipment, such as that described hereinafter.

The wet granules may thereafter be dried using standard techniques, such as under a current of dry air or, preferably, under vacuum at an elevated temperature (such as 30° C. or above). Drying should be to degree which results in greater than 0%, but less that 2%, w/w water content in the resultant granules. The dried granules are thereafter preferably handled in a manner that ensures that significant ingress of water is avoided prior to, and during, subsequent processing steps.

The dried granules are thereafter ground prior to extrusion. This separate grinding step is preferably performed by milling the dried granules in a ball mill, though any apparatus may be employed which results in the granules being broken down into particles of a smaller size.

Extrusion of the ground resultant may thereafter be conducted using standard extrusion equipment, for example a high pressure ram extruder or preferably a screw press, as described hereinafter. When the extruder that is employed is a screw extruder, exposure time in the extruder is from between 1 and 10 minutes, preferably between 4 and 6 minutes. The temperature profile preferably ranges from room temperature to 60° C. (e.g., 50° C.) on entering the extruder to no more than 120° C. (e.g., 110° C.) on leaving the extruder. Appropriate screw speeds are in the range 8 to 12 rpm, such as 10 rpm.

Compositions, and in particular implants, that are produced by way of the process of the invention may be used to treat/prevent diseases/conditions in mammalian patients depending upon the therapeutic agent (s) which is/are employed. For the above-mentioned drugs, diseases/conditions which may be mentioned include those against which the therapeutic agent (s) in question are known to be effective, and include those specifically listed for the drugs in question in Martindale, “The Extra Pharmacopoeia”, 31st Edition, Royal Pharmaceutical Society (1996). In particular, when compositions include leuprorelin, implants produced by way of the process of the invention may be used in contraception, as well as in the treatment of endometriosis, fibroids, benign prostate hypertropy, precocious puberty and/or cancer, such as breast cancer and, particularly, prostate cancer. We have found, surprisingly, that implants produced by way of the process of the invention comprising leuprorelin provide for an unexpectedly delayed castration in human subjects. Thus, implants obtainable by way of the process of the invention may allow for extended castration with low doses or leuprorelin when the latter is employed as active ingredient.

Implants produced by way of the process of the invention may be administered to patients by e.g., subcutaneous injection using standard techniques or, preferably, using a syringe as described in European patent No. EP 749,336 or U.S. Pat. No. 5,810,769. More than one implant may be administered to (or present in) a patient at any one time depending on the characteristics of the implant and the nature of the condition (s) that it is/are intended to treat.

The process of the invention is thus useful in the production of inter alia subcutaneous implants that may provide for extended release (i.e., continuously over a period of at least 3 to 6 months) of active ingredients, e.g., peptides, to mammalian patients. The process of the invention may also have the advantage that it may make use of established pharmaceutical processing methods, and employ materials that are approved for use in foods or pharmaceuticals or of like regulatory status.

The process of the invention may also possess the surprising advantage that implants produced thereby may provide for a pharmaceutically more beneficial release profile (e.g., a more extended, more controlled and/or more constant profile) than implants prepared by way of processes described in the prior art. The process of the invention may also provide the advantage that it may be used to prepare implants with a wider variety of active ingredients than may employ more standard procedures and/or otherwise be more conveniently performed by the skilled person than, processes described in the prior art for the preparation of subcutaneous implants.

### EXAMPLE

The invention is illustrated, but in no way limited, by the following example.

### Example 1

### Preparation of Implants Comprising Leuprorelin Acetate

Preparation of Equipment

Relevant production equipment was cleaned and sterilized. An isolator, equipment that was to be used inside the isolator for the manufacturing process, and raw materials, were sterilized in accordance with standard procedures (e.g., cloths soaked in absolute ethanol, isopropyl alcohol or SOPROPER® (aqueous peracetic acid; 3.5% w/w) and/or, in the case of equipment that is heat resistant, placing into heat-sealed plastic bags and heating to 150° C. for 2 hours).

Preparation of 'PLG

The temperature of the cryostat of an IKA 20 grinding mill was adjusted to 12° C. and allowed to stabilize. A 100 mL beaker was labelled and tared. 30 g of crude PLGA (Purac (Netherlands); particle size >150 μm; 75:25 lactide to glycolide unit ratio) was weighed into the beaker and the powder poured into the grinding mill. 3×1 mL of ethanol was distributed evenly over the PLGA by pipette. The grinding mill bowl was covered with a small cover and grinding commenced for 30 seconds, followed by a rest of 1 minute before opening and scraping up the dispersed powder. Further grinding was undertaken for 1 more minute and the subsequent procedure repeated. This was followed by grinding for 6 minutes and a repeat of the subsequent procedure.

The ground PLGA was then sieved by assembling the base of a standard sifting machine, and a 50 μm sieve and a 150 μm sieve, placing the ground PLGA onto the 150 μn sieve, attaching the cover, securing the whole, and adjusting the parameters on the sifting machine to give an interval time of 10 seconds, a sifting time of 3 and an amplitude of 2. The 50 to 150μ fraction was then collected in a tared flask and the <50 μm fraction and >150 μm fractions in two other flasks.

(Collected >150 μm fractions may be re-ground for a second time under the following conditions: weighing 30 g of >150 μm fraction(s) into a beaker and pouring the powder into the grinding mill, pipetting 1.5 mL of ethanol and distributing it evenly over the PLGA, covering the grinding mill bowl with a large cover, grinding for 3 minutes, and waiting for 1 minute before opening and scraping up the dispersed powder. The re-ground powder may then be sieved as described hereinbefore and re-ground again as necessary.) 50 to 150 μm fractions were dried by heating in an oven at 30° C., under a vacuum of −700 mm Hg for 24 hours (a transfer box was utilized to transfer the PLGA particles between the isolator and the vacuum oven). The dry PLGA was then placed into a flask in the isolator. (An analytical check was performed to check for residual ethanol.) Preparation of the Peptide Leuprorelin acetate (Bachem (Switzerland); 15 g) was densified using a Pulverisette monoplanetary grinding mill (Laval Labs Inc.). Three balls measuring 30 mm in diameter were placed into a 500 mL jar. The peptide was then poured carefully into the jar. The seal and cover were placed onto the jar and the jar placed onto its stand. The counterweight on the Pulverisette was adjusted to 4.6 kg. The rotation speed was set to 150 rpm for a milling time of 3 minutes.

Mixing The cover was thereafter removed from the jar and the pre-prepared PLGA was added to the peptide in the jar in an amount to give a mixture of 1:3 leuprorelin (calculated as the free base) to PLGA (w/w; i.e., 25% leuprorelin w/w). The seal and cover were placed onto the jar and the jar placed onto its stand. The counterweight on the Pulverisette was adjusted to 4.6 kg.

The rotation speed was set to 150 rpm for a milling time of 2 minutes, followed by a pause of 1 minute and a reverse milling time of 1 minute, followed by a repetition of the procedure.

A brown glass flask was labelled and tared. The densified mixture was transferred to the flask and the quantity of mixture noted. The flask was then removed from the isolator via the transfer chamber.

The flask containing the mixture was then secured onto a Turbula™ mixer (WAB). The Turbula speed was adjusted to 45 rpm and allowed to run for 15 minutes. The flask was then transferred back to the transfer chamber for exterior sterilization prior to wet granulation.

Wet Granulation of the Mixture

A K tool was fitted onto a Kenwood Mixer. The dry mixture from the previous step was carefully poured into the mixer's bowl. Water was added in a total amount that was proportional to the quantity of the leuprolide/PLGA mixture to be granulated (22% volume:weight of mixture), firstly by adding ⅔ of the volume of water to the mixture, adjusting the mixer to position 1, mixing for 1 minute, scraping the bottom of the mixer and the tool, and then by adding the remaining ⅓ of the water to the mixture, mixing for a further 2 minutes, and scraping the bottom of the mixer and the tool.

A tray from the transfer box covered in a sheet of aluminum foil was placed under an Erweke granulator. The granulator speed was adjusted to 60. The contents of the mixer bowl were placed onto a 1.6 mm screen in the granulator and granulation commenced. The granulated powder was collected on the tray.

Drying the Granules

The tray containing granules was placed back into the transfer box, which was then closed and removed from the isolator via the transfer chamber of the isolator. The transfer box was placed inside a solvent oven, pre-heated to a temperature of 30° C. A vacuum of −700 mm Hg was applied. Drying was allow to proceed for approximately 12 hours.

The transfer box was then returned to the transfer chamber for exterior sterilization.

Grinding the Dry Granules

3 balls measuring 30 mm in diameter were placed into the 500 mL jar of the Pulverisette grinding mill. The dry granules were then poured carefully into the jar. The seal and cover were placed onto the jar and the jar placed onto its stand. The counterweight on the Pulverisette was adjusted to 4.6 kg.

The rotation speed was set to 150 rpm for a milling time of 3 minutes.

A brown glass flask was labelled and tared. The ground mixture was collected in the flask, which was weighed and the mass of mixture noted.

Samples of the dry ground mixture were analyzed for water content (Karl Fischer), particle size, density, uniformity and leuprorelin content.

Subsequent Processing

The ground resultant was extruded into thin cylindrical shapes using a Scamia screw extruder. The extruder screw number was 190, screw speed 10 rpm and die number 4. The heating temperatures in the extruder were as follows: water bath 50° C.; Zone 1−70° C.; Zone 2−90° C.; Zone 3−110° C.

The extrudate was cut every 1.5 m or so. The diameter of the extrudate was measured using a Zumbasch laser in order to select the sections whose diameter conforms to the following specifications: For implants of diameter 1.6 mm±5%: minimum diameter 1.52 mm, maximum diameter 1.68 mm. For implants of diameter 1.8 mm±5%: minimum diameter 1.71 mm, maximum diameter 1.89 mm.

The density, uniformity of content, leuprorelin content and molecular weight of the extrudate was determined using standard techniques. On the basis of the analytical results, the length of the implant (to which the extrudate should be cut) was calculated using the formula below:

$$L = \frac{Dose \times 100}{3.14 \times {r}^{2} \times {T}_{m} \times {d}_{m} \times 0.985}$$

where r is the radius of the implant Tₘ is the average content (core loading percentage of peptide free base) and dₘ is the average density.

Thus, implants comprising 22.5 mg of leuprorelin (as the free base) were cut from extrudate with an approximate diameter of 1.6 mm to a length of approximately 35 mm. Each implant weighed approximately 90 mg and included between 23.6 and 26.2 mg of leuprorelin acetate. Similarly, implants comprising 30 mg of leuprorelin (as the free base) were cut from extrudate with an approximate diameter of 1.8 mm to a length of approximately 37 mm. Each implant weighed approximately 120 mg and included between 31.4 and 35 mg of leuprorelin acetate.

The implants were loaded into the needles of syringes as described hereinbefore, and packaged in an aluminum pouch in the presence of a desiccant bag. The aluminum pouch was then heat-sealed and sterilized by irradiation.

Other implants were made analogously to the process described above with the following dimensions: implants comprising 22.5 mg of leuprorelin (as the free base), an approximate diameter of 1.6 mm and a length of approximately 33 mm; implants comprising 27.5 mg of leuprorelin (as the free base), an approximate diameter of 2.0 mm and a length of approximately 26.6 mm.

What is claimed is:

1. A process for the preparation of an implantable or injectable pharmaceutical composition suitable for the extended release of an active ingredient to a patient following administration, which process comprises: (a) wet granulation of a mixture of an active ingredient and a copolymer of lactic acid and glycolic acid to form granules; (b) drying the granules; (c) grinding the dried granules to a powder; and (d) extruding the ground powder to form the composition.

2. The process of claim 1 wherein the active ingredient is a peptide or a peptide analogue.

3. The process of claim 1 wherein the extruded product is subsequently cut into lengths so as to form implants for intramuscular or subcutaneous administration.

4. The process of claim 3 wherein the length of the implants is about 10 to 60 mm.

5. The process of claim 4 wherein the length of the implants is about 20 to 40 mm.

6. The process of claim 1 which further comprises packaging the extruded product wherein the extruded product is sterilized before or after packaging.

7. The process of claim 6 which further comprises packaging the implants into syringe needles.

8. The process of claim 1 wherein the extruded product has a diameter of between about 1 and 3 mm.

9. The process of claim 8, wherein the extruded product has a diameter of between about 1.5 and 2 mm.

10. The process of claim 1, wherein the copolymer has a molar ratio of lactic acid to glycolic acid monomers of about 70:30 to about 80:20.

11. The process of claim 1, wherein the copolymer is provided in the form of particles having sizes of about 50 μm to about 150 μm.

12. The process of claim 1, wherein the active ingredient is GnRH (LHRH), a growth hormone releasing hormone, a growth hormone releasing peptide, angiotensin, bombesin, bradykinin, cholecystokinin, enkephalin, neurokinin, tachykinin, or an agonist or an antagonist of a receptor thereof.

13. The process of claim 1, wherein the active ingredient is a renin inhibitor, a protease inhibitor, a metallopeptidase inhibitor, an enkephalinase inhibitor, or an atrial or brain natriuretic factor degrading enzyme inhibitor.

14. The process of claim 13, wherein the active ingredient is buserelin, deslorelin, histrelin, avorelin, tryptorelin, goserelin, leuprorelin, cetrorelix, teverelix, ramorelix, antide, nictide, azaline B, azaline C, ganirelix or hexarelin.

15. The process of claim 14, wherein the active ingredient is leuprorelin or a pharmaceutically acceptable salt thereof.

16. The process of claim 15, wherein the active ingredient is leuprorelin acetate.

17. The process of claim 16, wherein the leuprorelin (calculated as the weight of the free base, excluding any weight resulting from the presence of a counter ion) and polymer are present in a weight ratio of about 1:3 w/w.

18. The process of claim 1, wherein the active ingredient is pre-treated in a ball mill prior to the wet granulation step.

19. The process of claim 1, wherein the wet granulation step employs water.

20. The process of claim 19, wherein the volume of water employed is about 22% of the total weight of the active ingredient/polymer mixture.

21. The process of claim 1, wherein the drying step is carried out under vacuum at above room temperature.

22. The process of claim 1, wherein the dried granules comprise greater than 0%, but less than 2%, w/w water.

23. The process of claim 1, wherein the grinding step involves milling the dried granules in a ball mill.

24. The process of claim 1, wherein the extrusion step is conducted using a screw press.

25. A product obtainable by the process of claim 1.

26. The product of claim 25 which provides an extended release of the active ingredient over a period at least three to six months.
